(19) United States
(12) Patent Application Publication
(10) Pub. No.: US 2024/0304277 A1
(43) Pub. Date: Sep. 12, 2024

(54) Forensic DNA Mixture Interpretation with Single-Cell Profiling

(71) Applicant: UNIVERSITY OF NORTH TEXAS HEALTH SCIENCE CENTER AT FORT WORTH, Fort Worth, TX (US)

(72) Inventors: Jianye Ge, Fort Worth, TX (US); Bruce Budowle, Fort Worth, TX (US); Jonathan King, Fort Worth, TX (US); Amy Smuts, Fort Worth, TX (US)

(21) Appl. No.: 18/263,906

(22) PCT Filed: Mar. 14, 2022
(86) PCT No.: PCT/US2022/020140

§ 371 Date: Aug. 2, 2023

Related U.S. Application Data
(60) Provisional application No. 63/161,721, filed on Mar. 16, 2021.

(51) Int. Cl. G16B 20/00; C12Q 1/6851

(52) U.S. Cl. CPC G16B 20/00; C12Q 1/6851

(57) ABSTRACT

The present invention includes methods and kits for determining one or more nucleic acid contributors to a sample or specimen from single cells in a sample, comprising: counting isolated cells and cell types; determining a mixture ratio of isolated cells and cell types; generating amplicons from each cell in the sample; calculating from the counted isolated cells, the cell types, the mixture ratio and amplicons from each single cell; comparing the amplicons a reference or known amplicon profile from a subject suspected of contributing nucleic acids; clustering the cells to contributors; identifying a number of contributors to the biological sample or specimen; generating consensus profiles for each contributor, and comparing the consensus profde of each contributor to a reference or known amplicon profde from a subject suspected of contributing nucleic acids to the biological sample or specimen.

### TECHNICAL FIELD OF THE INVENTION

The present invention relates in general to the field of forensics, and more particularly, to interpretation with single-cell profiling from forensic samples having complex mixtures.

### STATEMENT OF FEDERALLY FUNDED RESEARCH

Not applicable.

### INCORPORATION-BY-REFERENCE OF MATERIALS FILED ON COMPACT DISC

Not applicable.

### BACKGROUND OF THE INVENTION

Without limiting the scope of the invention, its background is described in connection with DNA mixtures.

Interpreting DNA mixtures is one of the most challenging problems in forensics genetics. The current standard workflow and mixture interpretation process are to extract DNA from a crime scene sample (e.g., swabs), quantify the extracted DNA, amplify targeted Short Tandem Repeat (STR) regions, detect DNA fragments through Capillary Electrophoresis (CE), call alleles with accompanying software, and interpret or deconvolve as best as is possible the collection of allelic peaks in an electropherogram primarily by a DNA analyst(s) based on training and experience with or without the assistance of probabilistic genotyping software programs. With this generalized CE-STR analysis process, although the DNA may still be contained in individual cells when collected, during extraction, the cells, and thus the DNA, are pooled. If there is more than one contributor to the sample, a mixture is observed. Subsequent to generating the mixture profile, an analyst(s) attempts to decipher the information to determine, for example, the number of contributors (NOC) in a mixture, the genotypes of individual contributors, if a particular individual is or is not a contributor of a mixture, etc. Given the available information generated through this standard process, the mixture profile is usually interpreted by indirect methods, such as inferring the NOC by counting the observed alleles or evaluating the weight of the evidence assuming a person as being a contributor vs. an unknown person being a contributor by a likelihood ratio (LR) approach. The LR compares the likelihoods of observing the same evidence given two or more competing hypotheses (e.g., the mixture is composed of the victim and the suspect vs. the mixture is composed of the victim and a random person in a population).

At times, the deconvolution of the contributing genotypes can be very challenging because of overlapping alleles, allele drop out (ADO), allele drop in (ADI) and uncertainty in the NOC. For example, it can be formidable with the current standard analysis to determine the NOC of a trio mixture formed by both parents and their child, since both alleles of the child are shared with the parents. In addition, it is difficult to determine if a peak (or signal) in a stutter position of a major contributor allele is composed of an allele from a minor contributor and stutter or solely a stutter product. Thus, some details of a mixture may not be able to be ascertained with a high degree of certainty.

One such invention is U.S. Pat. No. 9,637,799, issued to Fan et al., which is directed to massively parallel single cell analysis. These inventors are said to teach methods, compositions, and kits for multiplex nucleic acid analysis of single cells for use in massively parallel single cell sequencing. The methods, compositions and systems may be used to analyze thousands of cells concurrently, which cells may comprise a mixed population of cells (e.g., cells of different types or subtypes, different sizes).

However, despite these technologies, a need remains for the identification of contributors to a forensic sample, specifically, how to distinguish haploid and diploid profiles and methods to differentiate related contributors.

### SUMMARY OF THE INVENTION

In one embodiment, the present invention includes a method for determining one or more nucleic acid contributors to a biological sample or specimen from nucleic acids obtained from single cells in the biological sample or specimen, comprising the steps of: counting isolated cells and cell types in the biological sample or specimen; determining a mixture ratio of isolated cells and cell types in the biological sample or specimen; generating amplicons from each cell in the biological sample; calculating from the counted isolated cells, the cell types, the mixture ratio and amplicons from each single cell, the one or more nucleic acid contributors to the biological sample or specimen; comparing the amplicons from each cell to a reference or known amplicon profile from a subject suspected of contributing nucleic acids to the biological sample or specimen; and identifying a number of contributors to the biological sample or specimen. In one aspect, the method further comprises developing one or more consensus profile(s) of each nucleic acid contributor to the biological sample or specimen. In another aspect, the method further comprises calculating one or more consensus profile(s) to search against a DNA database profiles for matches (or hits). In another aspect, the method further comprises calculating one or more consensus profile(s) to search against a known profile to determine the nucleic acid contributor to the biological sample or specimen. In another aspect, the method further comprises, after the step of comparing the amplicons from each cell to a reference or known amplicon profile from a subject suspected of contributing nucleic acids to the biological sample or specimen, clustering each cell to a contributor. In another aspect, the method further comprises, after the step of identifying a number of contributors to the biological sample or specimen: generating consensus profiles for each contributor; and comparing the consensus profile of each contributor to a reference or known amplicon profile from a subject suspected of contributing nucleic acids to the biological sample or specimen. In another aspect, the nucleic acid contributor to the biological sample or specimen are obtained without mixing the nucleic acids from two or more cells. In another aspect, the method further comprises preparing at least one of a report with calculated profile from the subject suspected of contributing nucleic acids to the biological sample or specimen, or an indictment document against the subject identified as contributing nucleic acids to the biological sample or specimen. In another aspect, the nucleic acids comprise genomic DNA, and the method further comprises whole genome amplification. In another aspect, the nucleic acid comprises at least one of: a DNA a cDNA, a cDNA produced by reverse transcription of RNA, RNA, or the RNA is mRNA. In another aspect, the biological sample or specimen is obtained from a crime scene or crime victim. In another aspect a plurality of target-specific primer pairs are used for preamplification of one or more single nucleotide polymorphisms (SNPs) from each cell. In another aspect, an amplification is carried out by at least one of: polymerase chain reaction (PCR), a presence of an amplification product is determined by quantitative real-time polymerase chain reaction (qPCR), a universal qPCR probe is employed to detect amplification products, a universal qPCR probe comprises a double-stranded DNA (dsDNA) dye, or one or more target-specific qPCR probes is employed to detect amplification products. In another aspect, an amplification product is detected using capillary electrophoresis or a fluorogenic nuclease assay, or the presence of an amplification product is detected using a dual-labeled fluorogenic oligonucleotide probe. In another aspect, the single cells are obtained by at least one of: manual micromanipulation, Laser Capture Microdissection, Magnetic Activated Cell Sorting (MACS) flow cytometry, Fluorescent Activated Cell Sorting (FACS) flow cytometry, or dielectrophoresis. In another aspect, the mixture ratio of isolated cells and cell types in the biological sample or specimen is used to calculate allele drop-out (ADO) and allele drop-in (ADI) rates. In another aspect, at least 15, 20, 25, 30, 40, 50, 60, 75, 80, 90, 100, 125, 150, 160, 175, 200, 250, 300, 350, 400, 450, 500 or more cells are counted. In another aspect, the single cells are haploid or diploid cells. In another aspect, the nucleic acids from each cell are amplified, tagged and detected with a plurality of target nucleic acids; distributing a plurality of amplicons within a device comprising separate chambers; and amplifying and detecting the plurality of amplicons, wherein different amplicons are amplified and detected in separate chambers.

In another embodiment, the present invention includes a method of quantifying a nucleic acid sample comprising nucleic acid of one or more contributors, the method comprising: counting isolated cells and cell types in a biological sample or specimen; determining a mixture ratio of isolated cells and cell types in the biological sample or specimen; generating amplicons from each cell in the biological sample; calculating from the count of isolated cells, the cell types, the mixture ratio and amplicons from each single cell, with one or more processors, the one or more nucleic acid contributors to the biological sample or specimen; comparing, with the one or more processors, the amplicons from each cell to a reference or known amplicon profile from a subject suspected of contributing nucleic acids to the biological sample or specimen; clustering each of the cells to a contributor, and identifying a number of contributors to the biological sample or specimen; generating consensus profiles for each contributor; and comparing the consensus profile of each contributor to a reference or known amplicon profile from a subject suspected of contributing nucleic acids to the biological sample or specimen.

In another embodiment, the present invention includes a method, implemented at a computer system that includes one or more processors and system memory, of quantifying a nucleic acid sample comprising nucleic acid of one or more contributors, the method comprising: counting isolated cells and cell types in a biological sample or specimen; determining a mixture ratio of isolated cells and cell types in the biological sample or specimen; generating amplicons from each cell in the biological sample; calculating from the count of isolated cells, the cell types, the mixture ratio and amplicons from each single cell, with the one or more processors, the one or more nucleic acid contributors to the biological sample or specimen; comparing, with the one or more processors, the amplicons from each cell to a reference or known amplicon profile from a subject suspected of contributing nucleic acids to the biological sample or specimen; clustering the cells to contributors; and identifying, with the one or more processors, a number of contributors to the biological sample or specimen; generating consensus profiles for each contributor; and comparing the consensus profile of each contributor to a reference or known amplicon profile from a subject suspected of contributing nucleic acids to the biological sample or specimen. In one aspect, the method further comprises developing one or more consensus profile(s) of each nucleic acid contributor to the biological sample or specimen. In another aspect, the method further comprises calculating one or more consensus profile(s) to search against a DNA database profiles for matches (or hits). In another aspect, the method further comprises calculating one or more consensus profile(s) to search against a known profile to determine the nucleic acid contributor to the biological sample or specimen. In another aspect, the nucleic acid contributor to the biological sample or specimen are obtained without mixing the nucleic acids from two or more cells. In another aspect, the method further comprises preparing at least one of a report with calculated profile from the subject suspected of contributing nucleic acids to the biological sample or specimen, or an indictment document against the subject identified as contributing nucleic acids to the biological sample or specimen. In another aspect, the e nucleic acids comprise genomic DNA, and the method further comprises whole genome amplification. In another aspect, the nucleic acid comprising at least one of: a DNA a cDNA, a cDNA produced by reverse transcription of RNA, RNA, or the RNA is mRNA. In another aspect, the biological sample or specimen is obtained from a crime scene or crime victim. In another aspect, a plurality of target-specific primer pairs are used for preamplification of one or more single nucleotide polymorphisms (SNPs) from each cell. In another aspect, an amplification is carried out by at least one of: polymerase chain reaction (PCR), a presence of an amplification product is determined by quantitative real-time polymerase chain reaction (qPCR), a universal qPCR probe is employed to detect amplification products, a universal qPCR probe comprises a double-stranded DNA (dsDNA) dye, or one or more target-specific qPCR probes is employed to detect amplification products. In another aspect, the presence of an amplification product is detected using capillary electrophoresis or a fluorogenic nuclease assay, or the presence of an amplification product is detected using a dual-labeled fluorogenic oligonucleotide probe. In another aspect, the single cells are obtained by at least one of: manual micromanipulation, Laser Capture Microdissection, Magnetic Activated Cell Sorting (MACS) flow cytometry, Fluorescent Activated Cell Sorting (FACS) flow cytometry, or dielectrophoresis. In another aspect, the mixture ratio of isolated cells and cell types in the biological sample or specimen is used to calculate allele drop-out (ADO) and allele drop-in (ADI) rates. In another aspect, at least 15, 20, 25, 30, 40, 50, 60, 75, 80, 90, 100, 125, 150, 160, 175, 200, 250, 300, 350, 400, 450, 500 or more cells are counted. In another aspect, the single cells are haploid or diploid cells. In another aspect, the nucleic acids from each cell are amplified, tagged and detected with a plurality of target nucleic acids; distributing a plurality of amplicons within a device comprising separate chambers; and amplifying and detecting the plurality of amplicons, wherein different amplicons are amplified and detected in separate chambers.

In another embodiment, the present invention includes a kit for determining one or more nucleic acid contributors to a biological sample or specimen from nucleic acids obtained from single cells in the biological sample or specimen, comprising: a container comprising a plurality of primers selected to generate amplicons from single cells and reagents that generate amplicons from single cells in the biological sample or specimen; and instruction to: count isolated cells and cell types in the biological sample or specimen; determine a mixture ratio of isolated cells and cell types in the biological sample or specimen; calculate from the counted isolated cells, the cell types, the mixture ratio and amplicons from each single cell, the one or more nucleic acid contributors to the biological sample or specimen from amplicons found in the biological sample or specimen after amplification; and compare the amplicons from each cell to a reference or known amplicon profile from a subject suspected of contributing nucleic acids to the biological sample or specimen to identify a number of contributors to the biological sample or specimen.

### BRIEF DESCRIPTION OF THE DRAWINGS

For a more complete understanding of the features and advantages of the present invention, reference is now made to the detailed description of the invention along with the accompanying figures and in which:

FIG. 1 illustrates a mixture interpretation workflow with the single-cell mixture profiles.

FIG. 2 shows a mixture visualization example with 3D MDS plots.

FIGS. 3A and 3B show Identity-by-State (IBS) distributions of unrelated and related pairs (UR, PC, and FS, in dotted lines) and pairs of the true profile versus the consensus profile with a given number of cells (in solid lines) based on (3A) diploid cells and (3B) haploid cells. D=0.2; e=0.01; 1,000,000 simulations for each distribution. The IBS of “n cell(s)” is the IBS between true genotypes and the consensus genotypes from n cell(s). The consensus profile might contain missing alleles, and these missing alleles were excluded in counting IBS.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not delimit the invention, except as outlined in the claims.

As used herein, the term “amplification” refers to a method or reaction in which at least a part of at least one target nucleic acid is copied, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, any combinations thereof, such as, but not limited to: OLA/PCR, PCR/OLA, LDR/PCR, PCR/PCR/LDR, PCR/LDR, LCR/PCR, PCR/LCR, combined chain reaction (CCR), and the like. Descriptions of such techniques can be found in, among other sources, Ausbel et al.; PCR PRIMER: A LABORATORY MANUAL, Diffenbach, Ed., Cold Spring Harbor Press (1995); THE ELECTRONIC PROTOCOL BOOK, Chang Bioscience (2002); Msuih et al., J. Clin. Micro. 34:501-07 (1996); Innis et al., PCR PROTOCOLS: A GUIDE TO METHODS AND APPLICATIONS, Academic Press (1990), relevant portions incorporated herein by reference.

In some embodiments, amplification comprises at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally. In other embodiments, amplification includes isothermal amplification methods. Isothermal amplification uses a constant temperature rather than cycling through denaturation and annealing/extension steps. Some means of strand separation, e.g., an enzyme, is used in place of thermal denaturation.

For use with the present invention, amplicons can be produced upon preamplification and/or amplification, that are conveniently analyzed by an amplification method, such as PCR. In particular embodiments, as amplified sample from a single cell or small cell population may be used for many separate PCR reactions performed in a low-volume PCR reaction apparatus. In certain embodiments, preamplification is carried out using one or more primer pairs specific for the one or more target nucleic acids of interest. Thus, a low-volume PCR reaction apparatus can include separate reaction chambers for amplifying with each primer pair, such that the production of an amplicon in a particular reaction chamber indicates that the corresponding target nucleic acid was present in the sample.

Detection of amplicons is carried out using methods known in the art. These can include fluorometric methods, such as real-time quantitation method that monitoring the formation of amplification product involves the continuous measurement of PCR product accumulation using a dual-labeled fluorogenic oligonucleotide probe, e.g., a TaqMan® and U.S. Pat. No. 5,723,591, relevant portions incorporated herein by reference. TaqMan® is widely used for qPCR and the present invention is not limited to use of TaqMan® probes, but also, any suitable probes can be used with the present invention.

As used herein, the terms “biological sample” or “biological specimen” refers a biological fluid, tissue, residue or surface on which single cells or portions thereof can be obtained and are from a biological source. The samples or specimens are obtained and prepared using conventional methods known in the art. In particular, DNA or RNA are useful in the methods described herein and can be extracted and/or amplified from any source. Suitable nucleic acids can also be obtained from an environmental source (e.g., water), from man-made products (e.g., food), from forensic samples, and the like. Nucleic acids can be extracted or amplified from cells or portions thereof, bodily fluids (e.g., blood, a blood fraction, urine, feces, bodily secretions, etc.), or tissue samples by any of a variety of standard techniques. Non-limiting examples of samples or specimens include skin surfaces, genital areas or tracts, rectum, plasma, serum, spinal fluid, lymph fluid, peritoneal fluid, pleural fluid, oral fluid, samples from the respiratory, intestinal, genital, and urinary tracts; samples of tears, saliva, blood cells, from textiles (such as bedding or carpet), from door handles, etc. Samples can be obtained from live or dead organisms or processed products of organisms. Illustrative samples can include single cells, paraffin-embedded tissue samples, needle biopsies, and food products. Nucleic acids useful in the methods described herein can also be derived from one or more nucleic acid libraries, including cDNA, cosmid, YAC, BAC, P1, PAC libraries, and the like.

Nucleic acids of interest can be isolated using methods well known in the art, with the choice of a specific method depending on the source, the nature of biological sample or specimen, the nucleic acid, and environmental factors. The sample nucleic acids need not be in pure form but are typically sufficiently pure to allow the amplification steps of the methods described herein to be performed. Where the target nucleic acids are mRNA, the RNA can be reversed transcribed into cDNA by standard methods known in the art and as described in Sambrook, J., Fritsch, E. F., and Maniatis, T., Molecular Cloning: A Laboratory Manual. Cold Spring Harbor Laboratory Press, NY, Vol. 1, 2, 3 (1989), relevant portions incorporated herein by reference. cDNA can be analyzed according to the methods described herein.

As used herein, the term “hybridization” refers to the binding of a nucleic acid to a target nucleotide sequence in the absence of substantial binding to other nucleotide sequences present in the hybridization mixture under defined stringency conditions, such as low, medium, or high stringency. Those of skill in the art recognize that relaxing the stringency of the hybridization conditions allows sequence mismatches to be tolerated. In particular embodiments, hybridizations are carried out under stringent hybridization conditions as taught in, e.g., Berger and Kimmel (1987) METHODS IN ENZYMOLOGY, VOL. 152: GUIDE TO MOLECULAR CLONING TECHNIQUES, San Diego: Academic Press, Inc. and Sambrook et al. (1989) MOLECULAR CLONING: A LABORATORY MANUAL, 2ND ED., VOLS. 1-3, Cold Spring Harbor Laboratory), relevant portions incorporated herein by reference). The melting temperature of a hybrid (and thus the conditions for stringent hybridization) is affected by various factors such as the length and nature (DNA, RNA, base composition) of the primer or probe and nature of the target nucleic acid (DNA, RNA, base composition, present in solution or immobilized, and the like), as well as the concentration of salts and other components (e.g., the presence or absence of formamide, dextran sulfate, polyethylene glycol). The effects of these factors are well known and are discussed in standard references in the art. Illustrative stringent conditions suitable for achieving specific hybridization of most sequences are: a temperature of, e.g., at least about 65 degrees C. and a salt concentration of, e.g., 0.2 molar at pH7.

As used herein, the term “nucleic acid” refers to polynucleotides including natural nucleotides and nucleotide analogs that can function (e.g., hybridize) in a similar manner to naturally occurring nucleotides. The term nucleic acid includes any form of DNA or RNA, including, for example, genomic DNA; complementary DNA (cDNA), mRNA, other RNAs, DNA molecules produced synthetically or by amplification. The term nucleic acid also includes any chemical modification of the polynucleotides, such as by methylation and/or by capping. Nucleic acid modifications can include, e.g., chemical groups that incorporate additional charges, polarizability, hydrogen bonding, electrostatic interaction, and functionality to the individual nucleic acid bases, phosphodiester bonds, or to the nucleic acid as a whole. Nucleic acid(s) can be obtained a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the term “nucleotide tag” refers to a predetermined nucleotide sequence that is added to a target nucleotide sequence. The nucleotide tag can encode an item of information about the target nucleotide sequence, such the identity of the target nucleotide sequence, the chromosome from which that sequence derives, or the identity of the sample from which the target nucleotide sequence was derived. Nucleotide tag sequences are generally not used as primer binding sites in the first round of amplification.

As used herein, the term “oligonucleotide” refers to a polynucleotide that is relatively short, generally in the 15-25 range, but generally in the 20-30, 30-40, 40-50, 80, 90, 100, 125, 150, 175 or 200 nucleotide range. Typically, oligonucleotides are single-stranded DNA molecules, but double-stranded oligonucleotides can also be produced.

As used herein, the terms “polymorphic marker” or “polymorphic site” refer to a locus at which nucleotide sequence variance occurs. Illustrative markers have at least two alleles, each occurring at frequency of greater than 1% (lower percentages also are considered polymorphic), and more typically greater than 1% of a selected population. A polymorphic site can be as small as one base pair. Polymorphic markers include restriction fragment length polymorphism (RFLPs), variable number of tandem repeats (VNTR's), hypervariable regions, minisatellites, dinucleotide repeats, trinucleotide repeats, tetranucleotide repeats, pentanucleotide repeats, hexanucleotide repeats and beyond, simple sequence repeats, deletions, and insertion elements such as Alu. The first identified allelic form is arbitrarily designated as the reference form and other allelic forms are designated as alternative or variant alleles. The allelic form occurring most frequently in a selected population may sometimes be referred to as the wildtype form. Diploid organisms can be homozygous or heterozygous for allelic forms. A diallelic polymorphism has two forms, while a triallelic polymorphism has three forms and so on.

As used herein, the term “primer” refers to an oligonucleotide that is capable of hybridizing or annealing with a nucleic acid and serving as an initiation site for nucleotide (RNA or DNA) polymerization under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but primers are typically at least 6 nucleotides long and, more typically range from 10 to 30 nucleotides, or even more typically from 15 to 30 nucleotides, in length. Other primers can be somewhat longer, e.g., 30 to 50 nucleotides long. In this context, “primer length” refers to the length of an oligonucleotide or nucleic acid that hybridizes to a complementary “target” sequence and primes nucleotide synthesis. Short primer molecules generally require cooler temperatures to form sufficiently stable hybrid complexes with the template. A primer need not reflect the exact sequence of the template but must be sufficiently complementary to hybridize with a template.

As used herein, the term “primer site” or “primer binding site” refers to a segment of a target nucleic acid to which a primer hybridizes. A primer can include a nucleotide tag, e.g., appended to its 5′ end.

A primer is said to anneal to another nucleic acid if the primer, or a portion thereof, specifically hybridizes to a nucleotide sequence within the nucleic acid. The statement that a primer hybridizes to a particular nucleotide sequence is not intended to imply that the primer hybridizes either completely or exclusively to that nucleotide sequence.

As used herein, the term “primer pair” refers to a set of primers including a 5′-“upstream primer” or “forward primer” that hybridizes with the complement of the 5′-end of the DNA sequence to be amplified and a 3′-downstream primer (or reverse primer) that hybridizes with the 3′ end of the sequence to be amplified. As will be recognized by those of skill in the art, the terms “upstream” and “downstream” or “forward” and “reverse” are not intended to be limiting, but rather provide illustrative orientation in particular embodiments. A primer pair is said to be “unique” if it can be employed to specifically amplify a particular target nucleotide sequence in a given amplification mixture.

As used herein, the term “probe” refers to a nucleic acid capable of binding to a target nucleic acid of complementary sequence through one or more types of chemical bonds, generally through complementary base pairing, usually through hydrogen bond formation, thus forming a duplex structure. The probe binds or hybridizes to a “probe binding site.” The probe can be labeled with a detectable label to permit facile detection of the probe, particularly once the probe has hybridized to its complementary target. Alternatively, however, the probe can be unlabeled, but can be detectable by specific binding with a ligand that is labeled, either directly or indirectly. Probes can vary significantly in size. Generally, probes are at least 6 to 15 nucleotides in length. Other probes are at least 10, 15, 20, 25, 30, or 40 nucleotides long. Still other probes are somewhat longer, being at least 50, 60, 70, 80, or 90 nucleotides long. Yet other probes are longer still and are at least 100, 150, 200 or more nucleotides long. Probes can also be of any length that is within any range bounded by any of the above values (e.g., 15-20 nucleotides in length). Primers can also function as probes. Typically, the primer or probe can be perfectly complementary to the target nucleic acid sequence or can be less than perfectly complementary. In certain embodiments, the primer has at least 65% identity to the complement of the target nucleic acid sequence over a sequence of at least 7 nucleotides, more typically over a sequence in the range of 10-30 nucleotides, and often over a sequence of at least 14-25 nucleotides, and more often has at least 75% identity, at least 85% identity, at least 90% identity, or at least 95%, 96%, 97%. 98%, or 99% identity. It will be understood that certain bases (e.g., the 3′ base of a primer) are generally complementary to corresponding bases of the target nucleic acid sequence. Primer and probes typically anneal most specifically to the target sequence under stringent hybridization conditions.

As used herein, the term “qPCR” refers to quantitative real-time polymerase chain reaction (PCR), which is also known as “real-time PCR” or “kinetic polymerase chain reaction.”

As used herein, the term “reagent” refers broadly to any agent used in a reaction, other than the analyte (e.g., nucleic acid being analyzed). Illustrative reagents for a nucleic acid amplification reaction include, but are not limited to, buffer, metal ions, polymerase, reverse transcriptase, primers, nucleotides, labels, dyes, nucleases, and the like. Reagents for enzyme reactions include, for example, enzymes, substrates, cofactors, buffer, metal ions, inhibitors, and activators.

As used herein, the term “single nucleotide polymorphism” (SNP) refers to a polymorphic site occupied by a single nucleotide (although the nucleotides can be any number within a group), which is the site of variation between allelic sequences. The site is usually preceded and followed by highly conserved sequences of the allele (e.g., sequences that vary in less than 1/100 or 1/1000 members of the populations). A SNP usually arises due to substitution of one nucleotide for another at the polymorphic site. A transition is the replacement of one purine by another purine or one pyrimidine by another pyrimidine. A transversion is the replacement of a purine by a pyrimidine or vice versa. SNPs can also arise from a deletion of a nucleotide or an insertion of a nucleotide relative to a reference allele. In certain embodiments, a collection of SNPs, mRNAs, non-coding RNAs (e.g., miRNAs), etc., can be identified are used to determine the one or more nucleic acid contributors to a biological sample or specimen from nucleic acids obtained from single cells.

As used herein, the term “target-specific qPCR probe” refers to a qPCR probe that identifies the presence of an amplification product during qPCR, based on hybridization of the qPCR probe to a target nucleotide sequence present in the product.

Target nucleic acids can be amplified and can be detected using the methods described herein. In some embodiments, at least some nucleotide sequence will be known for the target nucleic acids. For example, if PCR is used for preamplification/amplification of target nucleic acids, sufficient sequence information is typically available for each end of a given target nucleic acid to permit design of suitable amplification primers, although, those of skill in the art appreciate that target nucleic acids of unknown sequence can be amplified (e.g., using a pool of degenerate primers or a pool of combinatorial primers, such as random hexamers) as can mRNA (e.g., using oligo-dT). Target nucleic acids include polymorphisms, such as single nucleotide polymorphisms (SNPs). In this case, the amplification primers can be SNP-specific, meaning that at least one primer hybridizes to a SNP, such that an amplicon is produced only if the SNP is present in the sample nucleic acids.

Typical thermal cycling devices and reactions can be used with the present invention such a fluorescent dyes that emit a light beam of a specified wavelength, and detectors that read the intensity of the fluorescent dye. Devices for use with the present invention include, but are not limited to devices that can include one or more of the following: a thermal cycler, light beam emitter, and a fluorescent signal detector, have been described, e.g., in U.S. Pat. Nos. 5,928,907; 6,015,674; and 6,174,670, relevant portions incorporated herein by reference. Thermal cycling and fluorescence detecting devices can be used for precise quantification of target nucleic acids. In some embodiments, fluorescent signals can be detected and displayed during and/or after one or more thermal cycles, thus permitting monitoring of amplification products as the reactions occur in “real-time.” In certain embodiments, one can use the amount of amplification product and number of amplification cycles to calculate how much of the target nucleic acid sequence was in the sample prior to amplification.

According to some embodiments, amplification products are monitored after a predetermined number of cycles to indicate the presence of the target nucleic acid sequence in the sample. One skilled in the art can easily determine, for any given sample type, primer sequence, and reaction condition, how many cycles are sufficient to determine the presence and quantity of a given target nucleic acid.

As used herein, the term “target nucleic acids” refers to specific nucleic acids to be detected, such as Short Tandem Repeat (STR), Single Nucleotide Polymorphisms (SNPs), Insertion-Deletions (Indels), sequences adjacent thereto, and the like. Target nucleic acids include, for example, loci of interest (STRs, SNPs, Indels). Target nucleic acids can also be RNA or DNA.

Non-coding RNAs include those RNA species that are not necessarily translated into protein. These include, but are not limited to, transfer RNA (tRNA) and ribosomal RNA (rRNA), as well as RNAs such as small nucleolar RNAs, microRNAs, small interfering RNAs, Piwi-interacting RNAs (piRNAs, particularly those in spermatogenesis), and long non-coding RNAs (long ncRNAs.

As used herein, the term “target nucleotide sequence” refers to a molecule that has the nucleotide sequence of a target nucleic acid, e.g., an amplification product obtained by amplifying a target nucleic acid or the cDNA produced upon reverse transcription of an mRNA target nucleic acid.

As used herein, the term a “complementary sequence” refers to polynucleotides with the capacity for binding between two nucleotides, e.g., a nucleotide at a given position is capable of hydrogen bonding with a nucleotide of another nucleic acid, then the two nucleic acids are considered to be complementary to one another at that position. As used herein, complementarity refers to traditional Watson-Crick or non-canonical pairing between two single-stranded nucleic acid molecules can be partial, in which only some of the nucleotides bind, or it can be complete complementarity when total sequence alignment exists between the single-stranded molecules. The degree of complementarity between nucleic acid strands has significant effects on the efficiency and strength of hybridization between nucleic acid strands and the consequent stacking interactions.

As used herein, the term “universal detection probe” refers to any probe that identifies the presence of an amplification product, regardless of the identity of the target nucleotide sequence present in the product.

As used herein, the term “universal qPCR probe” refers to any such probe that identifies the presence of an amplification product during qPCR. In certain embodiments, one or more amplification primers can comprise a nucleotide sequence to which a detection probe, such as a universal qPCR probe binds. In this manner, one, two, or more probe binding sites can be added to an amplification product during the amplification step of the methods described herein. Those of skill in the art recognize that the possibility of introducing multiple probe binding sites during preamplification (if carried out) and amplification facilitates multiplex detection, wherein two or more different amplification products can be detected in a given amplification mixture or aliquot thereof.

As used herein, the term “universal detection probe” refers to primers labeled with a detectable label (e.g., a fluorescent label), as well as non-sequence-specific probes, such as DNA binding dyes, including double-stranded DNA (dsDNA) dyes, such as SYBR Green.

The present inventors have found that interpreting DNA mixtures is one of the most challenging problems in forensics genetics. The current standard workflow and mixture interpretation process are to extract DNA from a crime scene sample (e.g., swabs), quantify the extracted DNA, amplify targeted Short Tandem Repeat (STR) regions, detect DNA fragments through Capillary Electrophoresis (CE), call alleles with accompanying software, and interpret or deconvolve as best as is possible the collection of allelic peaks in an electropherogram primarily by a DNA analyst(s) based on training and experience with or without the assistance of probabilistic genotyping software programs. With this generalized CE-STR analysis process, although the DNA may still be contained in individual cells when collected, during extraction, the cells, and thus the DNA, are pooled. If there is more than one contributor to the sample, a mixture is observed. Subsequent to generating the mixture profile, an analyst(s) attempts to decipher the information to determine, for example, the number of contributors (NOC) in a mixture, the genotypes of individual contributors, if a particular individual is or is not a contributor of a mixture, etc. Given the available information generated through this standard process, the mixture profile is usually interpreted by indirect methods, such as inferring the NOC by counting the observed alleles or evaluating the weight of the evidence assuming a person as being a contributor vs. an unknown person being a contributor by a likelihood ratio (LR) approach. The LR compares the likelihoods of observing the same evidence given two or more competing hypotheses (e.g., the mixture is composed of the victim and the suspect vs. the mixture is composed of the victim and a random person in a population). At times, the deconvolution of the contributing genotypes can be very challenging because of overlapping alleles, allele drop out (ADO), allele drop in (ADI) and uncertainty in the NOC. For example, it can be formidable with the current standard analysis to determine the NOC of a trio mixture formed by both parents and their child, since both alleles of the child are shared with the parents. In addition, it is difficult to determine if a peak in a stutter position of a major contributor allele is composed of an allele from a minor contributor and stutter or solely a stutter product. Thus, some details of a mixture may not be able to be ascertained with a high degree of certainty.

The known art, however, fails to provide methods to determine the number of contributors, the methods to generate consensus profile from the single-cell profiles in the same clusters, how to deal with haploid and diploid profiles differently, the methods to differentiate related contributors.

The recent single-cell detection and analysis technologies provide a greater impact on reducing the challenges of DNA mixture interpretation. No mixture is generated as each cell is independently analyzed and thus the data from a single cell are derived from a single contributor. The DNA quantity of the contributors of a mixture can be more precisely determined by counting the isolated cells and cell types; the mixture ratio can be easily determined even though ratios may not be necessary anymore; and the alleles of each single cell can be called independently. The interpretation of these results is fundamentally different from the current probabilistic genotyping interpretation but should be much simpler. In other words, instead of “guessing” the possible genotypes of the contributors as the probabilistic genotyping methods do, single-cell profiles can be directly compared with the reference/known profiles to support whether or not an individual is a contributor of a mixture. Therefore, this approach can reduce uncertainty regarding the profiles contributing to a mixture and thus provide stronger support for investigative leads and judicial decision making.

Although many studies have employed single-cell technologies for forensic applications, most of them were on the wet-lab work to generate genotype profiles from single cells. The inventors conducted the first study to develop a comprehensive interpretation workflow for single-cell profiles from mixtures are interpreted individually and then holistically, evaluate the capabilities of single-cell profiles for mixture interpretation, and support that the single-cell based mixture interpretation can provide a precision that cannot be achieved with current standard CE-STR analyses and probabilistic genotyping interpretation.

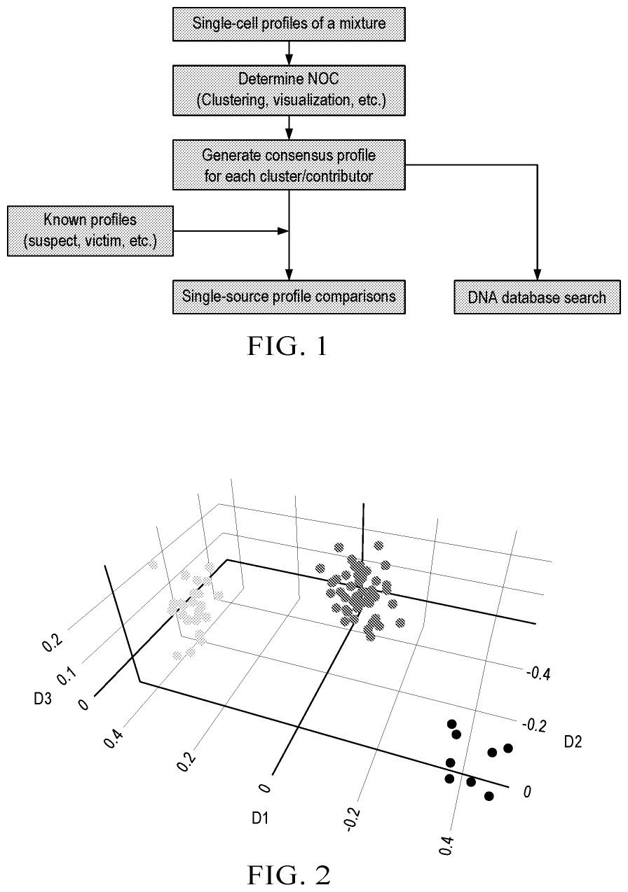

FIG. 1 illustrates a mixture interpretation workflow with the single-cell mixture profiles. First, with the single-cell profiles generated from a mixture, the NOC of the single-cell profiles is estimated, either by clustering (e.g., Expectation-Maximization (EM) algorithm and Silhouettes method), distance measure (e.g., Identity-by-State, IBS), or visualization (e.g., Multidimensional Scaling (MDS) plot). Second, the consensus profile of each cluster/contributor is generated from the single-cell profiles in each cluster. Then, the consensus profile(s) would be either directly searched against a DNA database(s) for partial or full matches (or hits), or compared with known profiles (e.g., suspect's profile) to determine if these profiles, or these profiles' relatives, potentially could be the contributors of the mixture.

With single-cell technologies, a DNA mixture is not formed during the analytical process. Thus, the interpretation is simplified and based on single source profile comparisons. Therefore, the methods employed in the probabilistic genotyping software program, as an indirect way to determine the genotypes of the contributors, may be unnecessary. Instead, the single-cell based approach can directly determine the profiles of the contributors clustered from single cells or even the single source profiles of the individual cells.

For the single source comparisons to confirm or exclude individuals, Identity-by-State (IBS) counting can be employed to determine the potential source or relationship between a consensus profile from single cells and a reference profile to be compared by a predefined IBS threshold. The traditional Likelihood Ratio (LR) based approach also can apply in these comparisons.

To reduce the limitations of current standard CE-STR based mixture analyses, alternate methods have been proposed, such as enzymatic digestion differential extraction (1, 2), reducing the allele overlap by using sequence-based alleles instead of traditional length-based alleles (3), employing probabilistic genotyping software in DNA profile interpretation (4-6), etc. While these alternate methods may add some value for mixture interpretation, the recent single-cell detection and analysis technologies provide a greater impact on reducing the challenges of DNA mixture interpretation.

In a single-cell analysis workflow, cells are individually isolated from forensic samples. There are several methods developed that are capable of isolating single cells, such as manual micromanipulation (7-13), Laser Capture Microdissection (LCM) (14, 15), Magnetic Activated Cell Sorting (MACS) flow cytometry (16), Fluorescent Activated Cell Sorting (FACS) flow cytometry (17-21), and a dielectrophoresis system (e.g., DEPArray) (22-24). After isolation, the single cells can be individually amplified for the targeted regions (e.g., STRs) or subjected to Whole Genome Amplification (WGA) before targeted amplification to enrich the DNA targets. Thus, the genotyping success rate could be improved (13, 25-30). Each of these devices and their methods of use are known to the skilled artisan following the manufacturer's instructions, which are incorporated herein by reference.

The present inventors recognized that single-cell technologies can recover the STR alleles of single cells with a high success rate. However, to date no study has provided a systematic approach to interpret the STR data from single cells derived from mixtures. The present invention shows the development of a comprehensive interpretation workflow and assessment of performance of interpretation methods for characterizing multiple donors from single-cell profiles of mixtures.

The present invention solves one or more of the following problems in the current art. (1) The mixture interpretation with single-cell profiles overcomes the forensic mixture problem. The interpretation methods with single-cell profiling are fundamentally different from those of the current CE-STR based analysis and the accompanying probabilistic genotyping interpretation approach. The current standard mixture interpretation is based on the CE-STR analysis, in which all cells from all contributors in a mixture are pooled in the whole workflow to generate a mixture profile. Subsequently, the mixture profile is interpreted to decipher the information and determine the genotypes of the individual contributors by a probabilistic genotyping approach. However, the single-cell profiling interpretation is based on the single-cell technologies, in which the individual cells are isolated and genotyped, the single-cell profiles are grouped into clusters by machine learning approaches, and then the single source profile for each cluster/contributor is generated by consensus. Subsequently, these consensus profiles can be either search against DNA database profiles for matches (or hits) or compared with known profiles to potentially determine their sources. (2) With the current CE-STR analysis, the deconvolution of the contributing genotypes can be very challenging because of overlapping alleles. For example, it is difficult to determine if a peak in a stutter position of a major contributor allele is composed of an allele from a minor contributor and stutter or solely a stutter product. However, mixture interpretation with single-cell profiling does not have allele overlapping issues, since all single-cell profiles are single source profiles. (3) The CE-STR mixture analysis and the probabilistic genotyping approach cannot interpret the mixtures with all contributors related (e.g., family trio contributors). However, these mixtures can precisely separate those contributors with single-cell profiling. (4) The CE-STR mixture analysis relies on a precise estimation of the mixture ratios of the contributors. However, the single-cell profiling mixture interpretation does not rely on the mixture ratios, although these ratios are available with the single-cell profiling. (5) The single-cell profiling mixture interpretation can easily give a precise estimation on the probability that a minor contributor during cell sampling is not detected. However, it is very challenging for CE-STR analysis to do so. (6) The mixture interpretation with single-cell profiles does not assume the independence among the tested markers, since the machine learning algorithms are able to accommodate dependent variants. Therefore, in contrast to the complexity of current practices to incorporate dependence among the markers, the single-cell based mixture interpretation can directly use the genetically linked markers and markers in Linkage-Disequilibrium (LD) together without any statistical corrections. Thus, the single cell-based interpretation can easily include as many markers as possible to increase the discriminating power and better identify individuals. However, it is very challenging for the current probabilistic genotyping approach to include dependent markers. (7) The single-cell based mixture interpretation is compatible with the current standard CODIS STRs and other commonly used forensic markers, such as Single Nucleotide Polymorphisms (SNPs), Insertion-Deletions (InDels), etc. Thus, the generated consensus profiles can be used for any relevant forensic applications. (8) The single-cell based mixture interpretation can utilize any genotype data generated from any genotyping or sequencing platforms, such as 3500 Genetic Analyzer, Illumina sequencers (e.g., MiSeq), MinION long-read sequencers, PacBio long-read sequencers, etc.

### Example 1. The Probability of Missing a Contributor During Sampling

Assuming the sampling process follows a binomial distribution and each cell has an equal chance to be sampled, the probability that none of the cells from a contributor with a mixture proportion of p is sampled in the total sampled n cells is (1−p)ⁿ. Table 1 estimates these probabilities for various mixture proportions of a contributor(s) and different total numbers of sampled cells, either diploid or haploid cells, assuming 6.6 pg DNA for a diploid cell and 3.3 pg DNA for a haploid cell.

TABLE 1
The probability of not detecting a minor contributor
in a set of single-cell samplings.

| No. of | DNA quantity | | The mixture proportion of a contributor | | | | |
|---|---|---|---|---|---|---|---|
| cells | Diploid | Haploid | 1% | 5% | 10% | 20% | 50% |
| 1 | 6.6 | 3.3 | 99.00% | 95.00% | 90.00% | 80.00% | 50.00% |
| 5 | 33 | 16.5 | 95.10% | 77.38% | 59.05% | 32.77% | 3.13% |
| 10 | 66 | 33 | 90.44% | 59.87% | 34.87% | 10.74% | 0.10% |
| 15 | 99 | 49.5 | 86.01% | 46.33% | 20.59% | 3.52% | 0.00% |
| 20 | 132 | 66 | 81.79% | 35.85% | 12.16% | 1.15% | 0.00% |
| 40 | 264 | 132 | 66.90% | 12.85% | 1.48% | 0.01% | 0.00% |
| 80 | 528 | 264 | 44.75% | 1.65% | 0.02% | 0.00% | 0.00% |
| 160 | 1056 | 528 | 20.03% | 0.03% | 0.00% | 0.00% | 0.00% |
| 500 | 3300 | 1650 | 0.66% | 0.00% | 0.00% | 0.00% | 0.00% |

### Example 2. The Accuracies of NOC Estimation

NOC estimation is the key component in this interpretation workflow. In general, NOC can be precisely determined with high number of sampled cells.

NOC estimation by clustering. The 2-person and 3-person mixtures were simulated with various numbers of cells, various mixture ratios, unrelated or related contributors, and both diploid and haploid cells. In the simulation, the ADO and ADI rates were set as 0.2 and 0.01, respectively, which were similar to the estimations of several studies and reflected the current performance of single-cell studies. For each mixture scenario, 10,000 cases were simulated, and the NOC for each case was estimated by the EM clustering algorithm and the Silhouettes method. Table 1 shows the accuracies of estimating the NOC for the simulated mixture scenarios.

Table 2. Accuracies of estimating the NOC with the EM algorithm and Silhouettes method for the various mixture scenarios. UR=unrelated, PC=parent-child, and FS=full-sibling. D=0.2; e=0.01; 10,000 simulations for each mixture scenario.

| | 2-person mixture | | | | | | | 3-person mixture | | | |
|---|---|---|---|---|---|---|---|---|---|---|---|
| No. of | | Diploid | | | Haploid | | | | Diploid | | Haploid |
| Cells | Mixture | UR | PC | FS | UR | PC | FS | Mixture | UR | Trio | UR |
| 160 | 80.80 | 100.00% | 100.00% | 99.90% | 99.90% | 98.04% | 92.16% | 53, 53, 53 | 99.96% | 88.32% | 96.55% |
| | 106.54 | 100.00% | 99.98% | 99.74% | 99.88% | 96.53% | 89.27% | 32, 32, 96 | 99.69% | 85.64% | 81.79% |
| | 128.32 | 100.00% | 99.85% | 98.87% | 99.16% | 91.35% | 81.70% | 16, 48, 96 | 98.60% | 78.46% | 52.55% |
| | 144.16 | 99.97% | 98.54% | 94.86% | 95.97% | 87.64% | 81.28% | | | | |
| | 152.8 | 99.55% | 91.59% | 86.12% | 92.43% | 87.98% | 83.97% | | | | |
| 80 | 40.40 | 100.00% | 99.93% | 99.91% | 99.93% | 98.73% | 96.32% | 26, 26, 26 | 99.97% | 87.94% | 96.44% |
| | 53.27 | 100.00% | 99.94% | 99.78% | 99.87% | 98.17% | 94.40% | 16, 16, 48 | 99.68% | 86.51% | 82.47% |
| | 64.16 | 99.99% | 99.96% | 99.53% | 99.71% | 94.37% | 87.68% | 8, 24, 48 | 98.36% | 80.54% | 54.15% |
| | 72.8 | 99.96% | 98.99% | 96.55% | 95.56% | 85.95% | 81.09% | | | | |
| | 76.4 | 99.67% | 93.47% | 88.56% | 88.91% | 83.54% | 79.10% | | | | |
| 40 | 20.20 | 100.00% | 99.99% | 99.93% | 99.72% | 95.25% | 92.41% | 13, 13, 13 | 99.96% | 88.29% | 91.81% |
| | 26.14 | 100.00% | 99.93% | 99.87% | 99.76% | 94.56% | 90.23% | 8, 8, 24 | 99.79% | 86.06% | 76.22% |
| | 32.8 | 100.00% | 99.89% | 99.22% | 99.18% | 89.52% | 81.95% | 4, 12, 24 | 98.44% | 79.79% | 46.55% |
| | 36.4 | 99.90% | 98.03% | 95.37% | 93.40% | 76.58% | 71.13% | | | | |
| | 38.2 | 95.49% | 78.98% | 74.69% | 84.31% | 68.47% | 65.89% | | | | |
| 20 | 10.10 | 100.00% | 99.54% | 99.02% | 97.21% | 82.30% | 79.50% | 6, 6, 6 | 99.81% | 85.59% | 69.19% |
| | 13.7 | 99.98% | 99.48% | 98.84% | 97.30% | 82.77% | 79.09% | 4, 4, 12 | 99.52% | 82.22% | 51.34% |
| | 16.4 | 99.96% | 99.62% | 98.92% | 95.34% | 73.85% | 70.30% | 2, 6, 12 | 89.80% | 72.16% | 21.69% |
| | 18.2 | 99.61% | 97.00% | 94.29% | 81.92% | 59.54% | 58.65% | | | | |
| | 19.1 | 98.51% | 86.74% | 82.46% | 60.82% | 53.45% | 53.40% | | | | |
| 10 | 5.5 | 99.95% | 96.77% | 94.02% | 82.78% | 56.59% | 55.61% | 3, 3, 3 | 97.16% | 66.81% | 33.58% |
| | 6.4 | 99.84% | 96.65% | 93.90% | 80.38% | 55.79% | 53.41% | 2, 2, 6 | 99.05% | 76.26% | 38.63% |
| | 8.2 | 99.68% | 94.61% | 91.60% | 78.59% | 49.60% | 48.24% | 1, 3, 6 | 59.31% | 37.65% | 12.44% |
| | 9.1 | 98.91% | 89.19% | 85.09% | 65.71% | 41.74% | 42.05% | | | | |

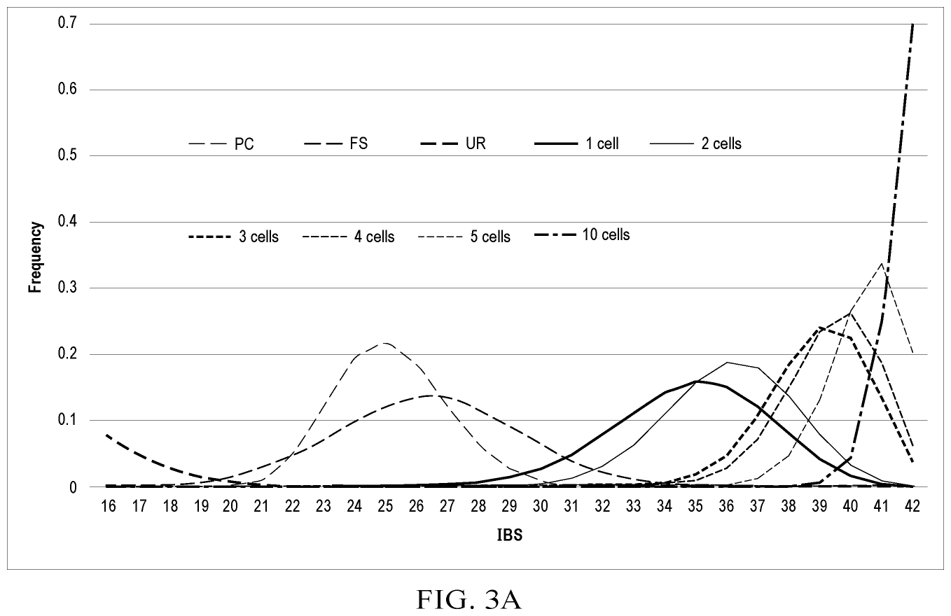

NOC estimation by visualization. NOC also can be estimated by visualization. Particularly when the number of sampled cells is small, visualization of the distance between and among single-cell profiles may facilitate determination of the NOC. FIG. 2 displays an MDS plot example for a family trio diploid cell mixture with 8, 24, 48 cells for father, mother, and child, respectively. All three contributors were clearly separated by clustering algorithm, which is not possible with the probabilistic genotyping interpretation with CE-STR analysis. FIG. 2 shows a mixture visualization example with 3D MDS plots.

NOC estimation by identity-by-state (IBS) distance measure. Another way to estimate the NOC is to measure the distances between single-cell profiles using predefined IBS thresholds. FIG. 2 shows the IBS distributions of unrelated and related pairs of individuals (UR=unrelated, PC=parent-child, and FS=fill-sibling) and pairs of the known alleles of a contributor versus the consensus alleles with a given number of sampled cells, assuming the clustered profiles were from the same contributor. Apparently, the IBS distance measure with predefined thresholds can precisely determine the NOC with haploid cells for unrelated contributors. if multiple cells could be associated by either clustering, measured distances, or visualization, the consensus profile could differentiate a profile from his/her parent or full-siblings simply by IBS counting, either with diploid or haploid cells.

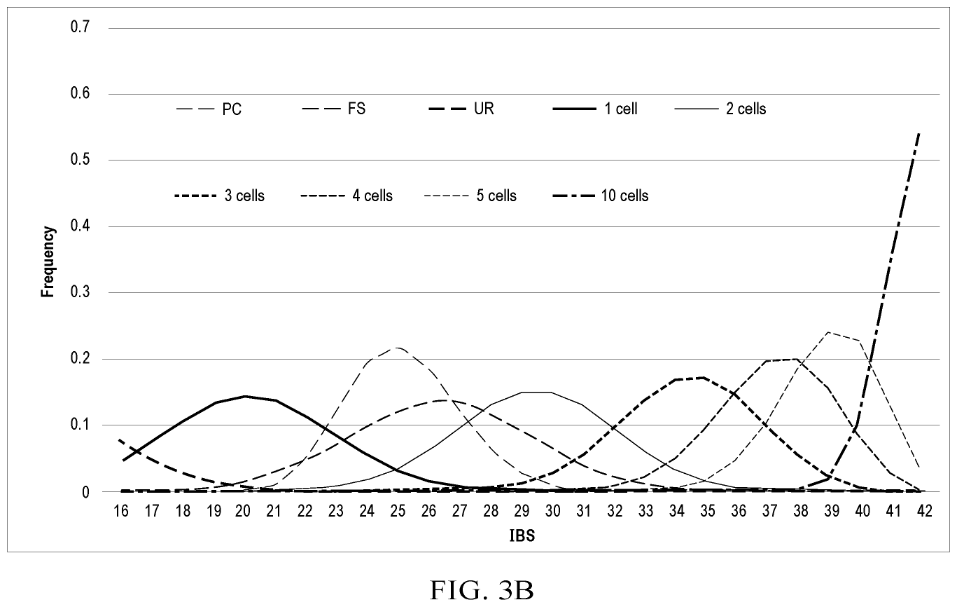

FIGS. 3A and 3B. IBS distributions of unrelated and related pairs (UR, PC, and FS, in dotted lines) and pairs of the true profile versus the consensus profile with a given number of cells (in solid lines) based on (FIG. 3A) diploid cells and (FIG. 3B) haploid cells. D=0.2; e=0.01; 1,000,000 simulations for each distribution. The IBS of “n cell(s)” is the IBS between true genotypes and the consensus genotypes from n cell(s). The consensus profile might contain missing alleles, and these missing alleles were excluded in counting IBS.

Accuracies of consensus. Once the NOC is estimated for a mixture, the consensus profile of each contributor can be generated by common alleles of the clustered single cells. With more cell profiles clustering in the same contributor, more shared alleles are found between the consensus profile and the true profile.

IBS distributions with consensus. As shown in FIG. 3A, one single diploid cell as a single contributor mostly shared at least 28 alleles (with an average of 35 alleles) with the true profile. Thus, there is sufficient precision to differentiate one single cell profile from a random person profile (˜0.03% average error rate with a threshold of IBS≤24; the average error rate was the average of the false positive rate of determining a consensus profile and an unrelated profile as from the same source, and the false negative rate of determining the two profiles as being from different sources). The consensus of haploid cell profiles would be more challenging. It is unlikely to determine the source of a single haploid cell profile by itself (FIG. 3B). However, the consensus profile of 2 haploid cells in a cluster, if they truly belong to the contributor, could be differentiated from a random person (<0.13% average error rate with a threshold of IBS≤21).

In certain cases, it may be necessary to determine if a suspect or one of the suspect's close relatives is a potential contributor of a mixture. Based on the IBS distributions in FIG. 3A, with only a few diploid cell profiles, an IBS threshold of ≤34 can precisely differentiate the related individuals if a consensus profile is from a known profile or a full-sibling of this known profile. With only 3 diploid cell profiles, 0.68% of the consensus profiles would have an IBS≤34 (i.e., false negatives), 0.18% of the FS pairs would have IBS≥35 (i.e., false positives or more appropriately adventitious associations). For haploid mixtures, if the same threshold is used (i.e., IBS≤34), 5 haploid cell profiles would be needed to achieve high accuracy (i.e., 0.64% false negative rate).

Accuracies of consensus by clustering. The distributions in FIGS> 3A and 3B assumed the cells clustered into contributors were truly the cells from this contributor. In fact, cells may be clustered to an incorrect contributor. However, the incorrectly clustered cells should be overwhelmed (assuming a sufficient number) by the cells correctly belonging to a contributor and the incorrect alleles should be filtered out by consensus, if a good clustering algorithm is used and there are enough number of cells clustered to this contributor.

To test this hypothesis, the consensus accuracies of clustering results were estimated, and the average accuracies of consensus alleles were calculated for each simulated mixture. Table 3 shows the average of 10,000 simulations for the average accuracies of the consensus profile. Only the cases with the correct NOC estimation were included in these calculations. For mixtures with an imbalanced mixture ratio (e.g., 1:3:6), the minor contributor(s) usually had lower consensus accuracies, mostly due to the small numbers of sampled cells.

As expected, given the correct NOC estimation, mixtures with a relatively high number of cells have very high consensus accuracies. Full profiles can be precisely obtained by consensus of the cells in the same clusters, if there are enough sampled diploid cells for a mixture with relatively balanced contributor ratios. Even with imbalanced contributors, all mixtures with at least 5 diploid cell profiles for the minor contributors achieved >96% average consensus accuracy, mostly greater than 97.6% (i.e., one allele mismatch or missing, 41/42). Particularly, the individual profiles of the family trio mixtures could be recovered even with only 40 sampled cells and imbalanced contributors, if one allele mismatch or missing was allowed.

The consensus accuracies of the haploid cell mixtures, in line with expectations, were lower than those of diploid cell mixtures. However, the accuracies were still high for mixtures with enough sampled cells (e.g., ≥20 cells) and relatively balanced contributors, even with related contributors (e.g., 94.51% for FS mixtures of 13:7).

Table 3. The average of the average accuracies of consensus alleles compared with the true alleles of the contributors. Only the mixtures with the correct NOC estimation were included. The NOC was determined using the EM algorithm and Silhouettes method. D=0.2; e=0.01; 10,000 simulations for each mixture scenario.

| | 2-person mixture | | | | | | | 3-person mixture | | | |
|---|---|---|---|---|---|---|---|---|---|---|---|
| No. of | | Diploid | | | Haploid | | | | Diploid | | Haploid |
| Cells | Mixture | UR | PC | FS | UR | PC | FS | Mixture | UR | Trio | UR |
| 160 | 80.80 | 100.00% | 100.00% | 100.00% | 100.00% | 99.93% | 99.28% | 53, 53, 53 | 100.00% | 100.00% | 100.00% |
| | 106.54 | 100.00% | 100.00% | 99.99% | 100.00% | 99.80% | 98.83% | 32, 32, 96 | 100.00% | 100.00% | 99.94% |
| | 128.32 | 100.00% | 99.98% | 99.90% | 99.55% | 98.64% | 97.17% | 16, 48, 96 | 99.96% | 99.95% | 99.47% |
| | 144.16 | 99.85% | 98.91% | 97.91% | 94.96% | 93.92% | 91.86% | | | | |
| | 152.8 | 98.01% | 90.16% | 89.05% | 81.77% | 85.70% | 84.88% | | | | |
| 80 | 40.40 | 100.00% | 100.00% | 100.00% | 100.00% | 99.96% | 99.73% | 26, 26, 26 | 99.99% | 99.99% | 99.97% |
| | 53.27 | 100.00% | 100.00% | 99.99% | 99.99% | 99.90% | 99.49% | 16, 16, 48 | 99.91% | 99.91% | 99.73% |
| | 64.16 | 99.94% | 99.93% | 99.89% | 99.75% | 99.09% | 97.90% | 8, 24, 48 | 99.47% | 99.45% | 98.53% |
| | 72.8 | 99.19% | 98.65% | 98.01% | 95.15% | 92.18% | 90.78% | | | | |
| | 76.4 | 96.40% | 93.09% | 91.63% | 81.77% | 83.45% | 83.02% | | | | |
| 40 | 20.20 | 99.96% | 99.96% | 99.96% | 99.93% | 99.80% | 99.52% | 13, 13, 13 | 99.69% | 99.68% | 99.00% |
| | 26.14 | 99.88% | 99.87% | 99.87% | 99.67% | 99.41% | 98.90% | 8, 8, 24 | 98.94% | 98.95% | 97.56% |
| | 32.8 | 99.21% | 99.18% | 99.08% | 98.37 | 96.61% | 95.43% | 4, 12, 24 | 97.79% | 97.78% | 94.55% |
| | 36.4 | 96.79% | 96.17% | 95.85% | 91.16% | 87.64% | 86.57% | | | | |
| | 38.2 | 85.79% | 81.23% | 81.22% | 76.48% | 78.91% | 78.88% | | | | |
| 20 | 10.10 | 99.16% | 99.17% | 99.13% | 98.20% | 96.75% | 95.68% | 6, 6, 6 | 96.98% | 96.97% | 92.68% |
| | 13.7 | 98.90% | 98.87% | 98.85% | 97.53% | 95.57% | 94.51% | 4, 4, 12 | 95.72% | 95.71% | 89.05% |
| | 16.4 | 96.83% | 96.80% | 96.71% | 93.02% | 89.51% | 88.51% | 2, 6, 12 | 94.02% | 94.02% | 84.83% |
| | 18.2 | 92.78% | 92.31% | 92.01% | 81.99% | 77.81% | 78.35% | | | | |
| | 19.1 | 90.85% | 88.80% | 87.86% | 69.30% | 68.57% | 69.78% | | | | |
| 10 | 5.5 | 96.33% | 96.34% | 96.30% | 91.56% | 87.57% | 86.61% | 3, 3, 3 | 92.68% | 92.74% | 78.80% |
| | 6.4 | 95.37% | 95.33% | 95.33% | 90.70% | 86.72% | 85.95% | 2, 2, 6 | 89.45% | 89.47% | 75.58% |
| | 8.2 | 92.11% | 91.99% | 91.97% | 82.58% | 78.46% | 77.78% | 1, 3, 6 | 90.86% | 90.42% | 66.12% |
| | 9.1 | 90.81% | 90.36% | 90.17% | 71.50% | 67.26% | 67.31% | | | | |

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Gill P, Jeffreys A J, Werrett D J. Forensic application of DNA ‘fingerprints’. Nature. 1985; 318(6046):577-9.
2. Voorhees J C, Ferrance J P, Landers J P. Enhanced elution of sperm from cotton swabs via enzymatic digestion for rape kit analysis. J Forensic Sci. 2006 May; 51(3):574-9. doi:10.1111/j.1556-4029.2006.00112.x.
3. Novroski N M M, King J L, Churchill J D, Seah L H, Budowle B. Characterization of genetic sequence variation of 58 STR loci in four major population groups. Forensic Sci Int Genet. 2016 November; 25:214-26. doi:10.1016/j.fsigen.2016.09.007.
4. Bright J A, Taylor D, McGovern C, Cooper S, Russell L, Abarno D, et al. Developmental validation of STRmix, expert software for the interpretation of forensic DNA profiles. Forensic Sci Int Genet. 2016 July; 23:226-39. doi:10.1016/j.fsigen.2016.05.007.
5. Gill P, Haned H, Eduardoff M, Santos C, Phillips C, Parson W. The open-source software LRmix can be used to analyse SNP mixtures. Forensic Sci Int Genet Suppl Ser. 2015; 5:e50-el.
6. Perlin M W, Legler M M, Spencer C E, Smith J L, Allan W P, Belrose J L, et al. Validating TrueAllele® DNA mixture interpretation. J Forensic Sci. 2011; 56(6):1430-47.
7. Findlay I, Taylor A, Quirke P, Frazier R, Urquhart A. DNA fingerprinting from single cells. Nature. 1997; 389(6651):555-6.
8. Li C, Qi B, Ji A, Xu X, Hu L. The combination of single cell micromanipulation with LV-PCR system and its application in forensic science. Forensic Sci Int Genet Suppl Ser. 2009; 2(1):516-7.
9. Huffman K, Hanson E, Ballantyne J. Recovery of Single Source DNA Profiles from Mixtures by Direct Single Cell Subsampling and Simplified Micromanipulation. Science & Justice. 2020. doi:10.1016/j.scijus.2020.10.005.
10. Xu C, Feng L, Yang F, Jia J, Ji A Q, Hu L, et al. Mucosal cell isolation and analysis from cellular mixtures of three contributors. J Forensic Sci. 2015 May; 60(3):783-6. doi:10.1111/1556-4029.12715.
11. Bruck S, Evers H, Heidorn F, Muller U, Kilper R, Verhoff M A. Single cells for forensic DNA analysis—from evidence material to test tube. J Forensic Sci. 2011 January; 56(1):176-80. doi:10.1111/j.1556-4029.2010.01553.x.
12. Schneider C, Müller U, Kilper R, Siebertz B. Low copy number DNA profiling from isolated sperm using the Aureka®-micromanipulation system. Forensic Science International: Genetics. 2012; 6(4):461-5. doi:10.1016/j.fsigen.2011.10.001.
13. Theunissen G M G, Rolf B, Gibb A, Jäger R. DNA profiling of sperm cells by using micromanipulation and whole genome amplification. Forensic Sci Int Genet Suppl Ser. 2017; 6:e497-e9. doi:10.1016/j.fsigss.2017.09.183.
14. Vandewoestyne M, Deforce D. Laser capture microdissection in forensic research: a review. Int J Legal Med. 2010; 124(6):513-21.
15. Han J P, Yang F, Xu C, Wei Y L, Zhao X C, Hu L, et al. A new strategy for sperm isolation and STR typing from multi-donor sperm mixtures. Forensic Sci Int Genet. 2014 November; 13:239-46. doi:10.1016/j.fsigen.2014.08.012.
16. Xu Y, Xie J, Chen R, Cao Y, Ping Y, Xu Q, et al. Fluorescence- and magnetic-activated cell sorting strategies to separate spermatozoa involving plural contributors from biological mixtures for human identification. Scientific Reports. 2016; 6(1). doi:10.1038/srep36515.
17. Schoell W M, Klintschar M, Mirhashemi R, Pertl B. Separation of sperm and vaginal cells with flow cytometry for DNA typing after sexual assault. Obstetrics & Gynecology. 1999; 94(4):623-7.
18. Verdon T J, Mitchell R J, Chen W, Xiao K, van Oorschot R A. FACS separation of non-compromised forensically relevant biological mixtures. Forensic Sci Int Genet. 2015 January; 14:194-200. doi:10.1016/j.fsigen.2014.10.019.
19. Miller J M, Brocato E R, Yadavalli V K, Greenspoon S A, Ehrhardt C J. Testing Hormone-specific Antibody Probes for Presumptive Detection and Separation of Contributor Cell Populations in Trace DNA Mixtures. bioRxiv. 2019.
20. Stokes N A, Stanciu C E, Brocato E R, Ehrhardt C J, Greenspoon S A. Simplification of complex DNA profiles using front end cell separation and probabilistic modeling. Forensic Science International: Genetics. 2018; 36:205-12. doi:10.1016/j.fsigen.2018.07.004.
21. Dean L, Kwon Y J, Philpott M K, Stanciu C E, Seashols-Williams S J, Cruz T D, et al. Separation of uncompromised whole blood mixtures for single source STR profiling using fluorescently-labeled human leukocyte antigen (HLA) probes and fluorescence activated cell sorting (FACS). Forensic Science International: Genetics. 2015; 17:8-16.
22. Williamson V R, Laris T M, Romano R, Marciano M A. Enhanced DNA mixture deconvolution of sexual offense samples using the DEPArray system. Forensic Sci Int Genet. 2018 May; 34:265-76. doi:10.1016/j.fsigen.2018.03.001.
23. Anslinger K, Graw M, Bayer B. Deconvolution of blood-blood mixtures using DEPArray™ separated single cell STR profiling. Rechtsmedizin. 2018; 29(1):30-40. doi:10.1007/s00194-018-0291-1.
24. Fontana F, Rapone C, Bregola G, Aversa R, de Meo A, Signorini G, et al. Isolation and genetic analysis of pure cells from forensic biological mixtures: The precision of a digital approach. Forensic Sci Int Genet. 2017 July; 29:225-41. doi:10.1016/j.fsigen.2017.04.023.
25. England R, Nancollis G, Stacey J, Sarman A, Min J, Harbison S. Compatibility of the ForenSeq™ DNA Signature Prep Kit with laser microdissected cells: An exploration of issues that arise with samples containing low cell numbers. Forensic Science International: Genetics. 2020:102278.
26. Lim H H, Srisiri K, Rerkamnuaychoke B, Bandhaya A. A comparative study of whole genome amplification and low-template DNA profiling. Forensic Sci Int Genet Suppl Ser. 2019; 7(1):509-11.
27. Deleye L, Vander Plaetsen A S, Weymaere J, Deforce D, Van Nieuwerburgh F. Short Tandem Repeat analysis after Whole Genome Amplification of single B-lymphoblastoid cells. Sci Rep. 2018 Jan. 19; 8(1):1255. doi:10.1038/s41598-018-19509-5.
28. Theunissen G M, Rolf B, Gibb A, Jäger R. DNA profiling of sperm cells by using micromanipulation and whole genome amplification. Forensic Sci Int Genet Suppl Ser. 2017; 6:e497-e9.
29. Schneider P, Balogh K, Naveran N, Bogus M, Bender K, Lareu M, et al., editors. Whole genome amplification—the solution for a common problem in forensic casework? International Congress Series; 2004. Elsevier.
30. Chen M, Zhang J, Zhao J, Chen T, Liu Z, Cheng F, et al. Comparison of CE- and MPS-based analyses of forensic markers in a single cell after whole genome amplification. Forensic Science International: Genetics. 2020; 45. doi:10.1016/j.fsigen.2019.102211.
31. Li C-x, Han J-p, Ren W-y, Ji A-q, Xu X-l, Hu L. DNA profiling of spermatozoa by laser capture microdissection and low volume-PCR. PLoS One. 2011; 6(8):e22316.
32. Chen C, Xing D, Tan L, Li H, Zhou G, Huang L, et al. Single-cell whole-genome analyses by Linear Amplification via Transposon Insertion (LIANTI). Science. 2017; 356(6334):189-94.

1. A method for determining one or more nucleic acid contributors to a biological sample or specimen from nucleic acids obtained from single cells in the biological sample or specimen, comprising the steps of:
counting isolated cells and cell types in the biological sample or specimen;
determining a mixture ratio of isolated cells and cell types in the biological sample or specimen;
generating amplicons from each cell in the biological sample;
calculating from the counted isolated cells, the cell types, the mixture ratio and amplicons from each single cell, the one or more nucleic acid contributors to the biological sample or specimen;
comparing the amplicons from each cell to a reference or known amplicon profile from a subject suspected of contributing nucleic acids to the biological sample or specimen; and
identifying a number of contributors to the biological sample or specimen.

2. The method of claim 1, further comprising at least one of: developing one or more consensus profile(s) of each nucleic acid contributor to the biological sample or specimen; calculating one or more consensus profile(s) to search against a DNA database profiles for matches (or hits);
comparing the amplicons from each cell to a reference or known amplicon profile from a subject suspected of contributing nucleic acids to the biological sample or specimen, clustering each cell to a contributor;
after the step of comparing the amplicons from each cell to a reference or known amplicon profile from a subject suspected of contributing nucleic acids to the biological sample or specimen, clustering each cell to a contributor;
after the step of identifying a number of contributors to the biological sample or specimen;
generating consensus profiles for each contributor; and
comparing the consensus profile of each contributor to a reference or known amplicon profile from a subject suspected of contributing nucleic acids to the biological sample or specimen; or
preparing at least one of a report with calculated profile from the subject suspected of contributing nucleic acids to the biological sample or specimen, or an indictment document against the subject identified as contributing nucleic acids to the biological sample or specimen.

3.-6. (canceled)

7. The method of claim 1, wherein the nucleic acid contributor to the biological sample or specimen are obtained without mixing the nucleic acids from two or more cells.

8. (canceled)

9. The method of claim 1, wherein the nucleic acids comprise genomic DNA, and the method further comprises whole genome amplification; or a cDNA produced by reverse transcription of RNA, RNA, or the RNA is mRNA.

10. (canceled)

11. The method of claim 1, wherein the biological sample or specimen is obtained from a crime scene or crime victim.

12. The method of claim 1, wherein at least one of: a plurality of target-specific primer pairs are used for preamplification of one or more single nucleotide polymorphisms (SNPs) from each cell;
an amplification is carried out by at least one of: polymerase chain reaction (PCR), a presence of an amplification product is determined by quantitative real-time polymerase chain reaction (qPCR), a universal qPCR probe is employed to detect amplification products, a universal qPCR probe comprises a double-stranded DNA (dsDNA) dye, or one or more target-specific qPCR probes is employed to detect amplification products;
a presence of an amplification product is detected using capillary electrophoresis or a fluorogenic nuclease assay, or the presence of an amplification product is detected using a dual-labeled fluorogenic oligonucleotide probe;
a mixture ratio of isolated cells and cell types in the biological sample or specimen is used to calculate allele drop-out (ADO) and allele drop-in (ADI) rates;
at least 15, 20, 25, 30, 40, 50, 60, 75, 80, 90, 100, 125, 150, 160, 175, 200, 250, 300, 350, 400, 450, 500 or more cells are counted; or
the single cells are haploid or diploid cells.

13.-14. (canceled)

15. The method of claim 1, wherein the single cells are obtained by at least one of: manual micromanipulation, Laser Capture Microdissection, Magnetic Activated Cell Sorting (MACS) flow cytometry, Fluorescent Activated Cell Sorting (FACS) flow cytometry, or dielectrophoresis.

16.-18. (canceled)

19. The method of claim 1, wherein the nucleic acids from each cell are amplified, tagged and detected with a plurality of target nucleic acids; distributing a plurality of amplicons within a device comprising separate chambers; and amplifying and detecting the plurality of amplicons, wherein different amplicons are amplified and detected in separate chambers.

20. A method of quantifying a nucleic acid sample comprising nucleic acid of one or more contributors, the method comprising:
counting isolated cells and cell types in a biological sample or specimen;
determining a mixture ratio of isolated cells and cell types in the biological sample or specimen;
generating amplicons from each cell in the biological sample;
calculating from the count of isolated cells, the cell types, the mixture ratio and amplicons from each single cell, with one or more processors, the one or more nucleic acid contributors to the biological sample or specimen;
comparing, with the one or more processors, the amplicons from each cell to a reference or known amplicon profile from a subject suspected of contributing nucleic acids to the biological sample or specimen;
clustering each of the cells to a contributor; and
identifying a number of contributors to the biological sample or specimen;
generating consensus profiles for each contributor; and
comparing the consensus profile of each contributor to a reference or known amplicon profile from a subject suspected of contributing nucleic acids to the biological sample or specimen.

21. A method, implemented at a computer system that includes one or more processors and system memory, of quantifying a nucleic acid sample comprising nucleic acid of one or more contributors, the method comprising:
counting isolated cells and cell types in a biological sample or specimen;
determining a mixture ratio of isolated cells and cell types in the biological sample or specimen;
generating amplicons from each cell in the biological sample;
calculating from the count of isolated cells, the cell types, the mixture ratio and amplicons from each single cell, with the one or more processors, the one or more nucleic acid contributors to the biological sample or specimen;
comparing, with the one or more processors, the amplicons from each cell to a reference or known amplicon profile from a subject suspected of contributing nucleic acids to the biological sample or specimen;
clustering the cells to contributors; and
identifying, with the one or more processors, a number of contributors to the biological sample or specimen;
generating consensus profiles for each contributor; and
comparing the consensus profile of each contributor to a reference or known amplicon profile from a subject suspected of contributing nucleic acids to the biological sample or specimen.

22. The method of claim 21, further comprising at least one of: developing one or more consensus profile(s) of each nucleic acid contributor to the biological sample or specimen;
calculating one or more consensus profile(s) to search against a DNA database profiles for matches (or hits);
calculating one or more consensus profile(s) to search against a known profile to determine the nucleic acid contributor to the biological sample or specimen; or
preparing at least one of a report with calculated profile from the subject suspected of contributing nucleic acids to the biological sample or specimen, or an indictment document against the subject identified as contributing nucleic acids to the biological sample or specimen.

23.-24. (canceled)

25. The method of claim 21, wherein the nucleic acid contributor to the biological sample or specimen are obtained without mixing the nucleic acids from two or more cells.

26. (canceled)

27. The method of claim 21, wherein the nucleic acids comprise genomic DNA, and the method further comprises whole genome amplification; or a cDNA produced by reverse transcription of RNA, RNA, or the RNA is mRNA.

28. (canceled)

29. The method of claim 21, wherein the biological sample or specimen is obtained from a crime scene or crime victim.

30. The method of claim 21, wherein a plurality of target-specific primer pairs are used for preamplification of one or more single nucleotide polymorphisms (SNPs) from each cell;
an amplification is carried out by at least one of: polymerase chain reaction (PCR), a presence of an amplification product is determined by quantitative real-time polymerase chain reaction (qPCR), a universal qPCR probe is employed to detect amplification products, a universal qPCR probe comprises a double-stranded DNA (dsDNA) dye, or one or more target-specific qPCR probes is employed to detect amplification products;
a presence of an amplification product is detected using capillary electrophoresis or a fluorogenic nuclease assay, or the presence of an amplification product is detected using a dual-labeled fluorogenic oligonucleotide probe;
a mixture ratio of isolated cells and cell types in the biological sample or specimen is used to calculate allele drop-out (ADO) and allele drop-in (ADI) rates;
at least 15, 20, 25, 30, 40, 50, 60, 75, 80, 90, 100, 125, 150, 160, 175, 200, 250, 300, 350, 400, 450, 500 or more cells are counted; or
the single cells are haploid or diploid cells.

31.-32. (canceled)

33. The method of claim 21, wherein the single cells are obtained by at least one of: manual micromanipulation, Laser Capture Microdissection, Magnetic Activated Cell Sorting (MACS) flow cytometry, Fluorescent Activated Cell Sorting (FACS) flow cytometry, or dielectrophoresis.

34.-36. (canceled)

37. The method of claim 21, wherein the nucleic acids from each cell are amplified, tagged and detected with a plurality of target nucleic acids; distributing a plurality of amplicons within a device comprising separate chambers; and amplifying and detecting the plurality of amplicons, wherein different amplicons are amplified and detected in separate chambers.

38. A kit for determining one or more nucleic acid contributors to a biological sample or specimen from nucleic acids obtained from single cells in the biological sample or specimen, comprising:
a container comprising a plurality of primers selected to generate amplicons from single cells and
reagents that generate amplicons from single cells in the biological sample or specimen; and
instruction to:
count isolated cells and cell types in the biological sample or specimen;
determine a mixture ratio of isolated cells and cell types in the biological sample or specimen;
calculate from the counted isolated cells, the cell types, the mixture ratio and amplicons from each single cell, the one or more nucleic acid contributors to the biological sample or specimen from amplicons found in the biological sample or specimen after amplification; and
compare the amplicons from each cell to a reference or known amplicon profile from a subject suspected of contributing nucleic acids to the biological sample or specimen to identify a number of contributors to the biological sample or specimen.
